(19) United States
(12) Patent Application Publication
(10) Pub. No.: US 2026/0335570 A1
(43) Pub. Date: Oct. 29, 2026

(54) DRY-POWDER INHALER

(71) Applicant: APTAR FRANCE SAS, LE NEUBOURG (FR)

(72) Inventor: Franck POULLAIN, LA HAYE MALHERBE (FR)

(73) Assignee: APTAR FRANCE SAS, LE NEUBOURG (FR)

(21) Appl. No.: 19/103,919

(22) PCT Filed: Aug. 16, 2023
(86) PCT No.: PCT/FR2023/051274

§ 371 Date: Feb. 14, 2025

(30) Foreign Application Priority Data
Aug. 18, 2022 (FR) FR2208366

(51) Int. Cl. A61M 15/00

(52) U.S. Cl. CPC A61M 15/0008; A61M 15/0021; A61M 15/003; A61M 2202/064; A61M 2205/583

(57) ABSTRACT

A dry-powder inhaler having a body containing a chamber and a receiving sleeve, a reservoir unit removably inserted into the receiving sleeve. The reservoir unit has (i) a reservoir containing a dose of dry powder and a lateral outlet opening. The reservoir is movable between a non-loaded position disposed in the receiving sleeve and a loaded position disposed entirely in the chamber; (ii) a closure ring movable relative to the reservoir between a closed position of the lateral outlet opening and an open position of the lateral outlet opening. The closure ring is in a closed position when the reservoir is in the non-loaded position and in an open position when the reservoir is in the loaded position; and (iii) an actuator member forming a mouthpiece, for moving the reservoir from its non-loaded position to its loaded position, the actuator member defining a dispensing orifice.

The present invention relates to a dry-powder inhaler.

Dry-powder inhalers are well known in the prior art. Various types exist.

A first type of multidose inhaler contains a reservoir receiving multiple doses of powder, the inhaler being provided with dosage means, making it possible, on each actuation, to separate out one dose of this powder from the reservoir in order to bring it into an expulsion duct so that it can be dispensed to the user. Such devices require complicated and therefore expensive dosage means, and dosage accuracy and reproducibility are not guaranteed. In addition, there are risks of contamination of the powder disposed in the reservoir as the inhaler is being used.

Another type of multidose inhaler consists of providing several individual reservoirs each containing one dose of powder, then opening one of those reservoirs each time the inhaler is actuated. That implementation ensures that the powder is better sealed, since each dose of the powder is only exposed to the atmosphere at the moment when it is expelled. In order to produce such sets of individual reservoirs, different variations have already been proposed, such as blister strips or disks. With regard to opening the individual reservoirs, peeling off or unsticking the closure layer of the blisters has been proposed. That suffers from the disadvantage of difficulty in controlling the forces to be applied in order to guarantee full opening, without running the risk of opening the next reservoir, particularly if the opening means need to be actuated by inhalation. Another solution is to pierce the closure membrane of a blister on each actuation, which requires complicated and therefore expensive piercing means.

In addition, the multidose inhalers described above suffer from the problem of not guaranteeing optimal dispensing effectiveness, with a significant portion of the dose effectively penetrating into the user's lungs in order to have a beneficial therapeutic effect.

In order to make devices less complicated and thus less expensive, single-dose inhalers have been proposed which comprise a single individual reservoir, such as a blister or a capsule, which has to be loaded into the inhaler just before it is used. The advantage of such single-dose devices is that it is not necessary to store all of the doses inside the device, so that the dimensions thereof can be reduced. In contrast, it is more complicated for the user to use, since they are obliged to load a blister or a capsule into the inhaler before each use. Furthermore, other disadvantages specific to these single-dose inhalers exist. Thus, a blister or capsule does not allow large doses, typically greater than 100 mg, to be dispensed. In addition, in particular as regard capsules or blisters to be pierced, opening requires the use of complicated piercing means, and also risks getting residues of pierced membrane in the powder which is dispensed. Furthermore, the emptying performance of the reservoir is not always optimal, since a portion of the powder could remain inside the reservoir during inhalation.

In an attempt to overcome these disadvantages, document WO 98/26828 proposes a single-dose inhaler comprising a cylindrical reservoir provided with a lateral opening, disposed in a cylindrical chamber with a larger diameter, in a manner such that during inhalation, the reservoir is displaced in the chamber in accordance with an orbital movement, which causes the powder to be expelled. This implementation effectively makes it possible to improve the efficiency of dispensing, with substantially complete emptying of the reservoir and a larger portion of the dose which penetrates effectively into the lungs. Nevertheless, a disadvantage with this device is that the lateral opening of the reservoir must be opened before the reservoir is placed in the device, which presents a risk of losing powder during this placing, or contamination of the latter, in particular if the device is not used quickly after placing the reservoir. In addition, if the inhaler is reused with another reservoir, the mouthpiece part may be polluted by the previous use, in particular the protective grid provided upstream from the dispensing orifice of the inhaler.

Documents WO2013008037A1, WO2012163704A2, WO2008001132A1 and WO9826828A2 describe other devices of the state of the art.

An object of the present invention is to provide a powder inhaler that does not have the above-mentioned drawbacks.

In particular, an object of the present invention is to provide an inhaler of this type that can easily be reused with several reservoirs, without any risk of pollution or contamination of the inhaler.

Another objective of the present invention is to provide an inhaler of this type which limits or even avoids the risks of contamination and/or pollution of the powder contained in the reservoir.

Another objective of the present invention is to provide an inhaler of this type which reduces the complexity of use for the user and which guarantees better safety in use.

Another objective of the present invention is to provide an inhaler of this type which is simple and inexpensive to manufacture and to assemble.

The present invention thus provides a dry-powder inhaler comprising:
a body containing a chamber and a receiving sleeve,
a reservoir unit intended to be removably inserted into said receiving sleeve, said reservoir unit comprising:
(i) a reservoir containing a dose of dry powder to be inhaled and comprising a lateral outlet opening, reservoir being movable in said inhaler between a non-loaded position in which it is disposed in said receiving sleeve, and a loaded position in which it is disposed entirely in said chamber,
(ii) a closure ring movable relative to said reservoir between a closed position of said lateral outlet opening and an open position of said lateral outlet opening, said closure ring being in a closed position when said reservoir is in the non-loaded position, and being in an open position when said reservoir is in the loaded position,
(iii) an actuator member forming a mouthpiece, for moving said reservoir from its non-loaded position to its loaded position, said actuator member defining a dispensing orifice of said inhaler.

Advantageously, said actuator member comprises a grid upstream from said dispensing orifice.

Advantageously, said chamber is disposed between an inhalation air inlet and said receiving sleeve.

Advantageously, the diameter of said reservoir is less than the diameter of said chamber.

Advantageously, tangential channels bring a flow of inhalation air into said chamber in a tangential manner, which generates an orbital displacement of said reservoir in said chamber.

Advantageously, said reservoir contains a dose of powder greater than 50 mg, in particular greater than 100 mg.

Advantageously, the volume occupied by said reservoir in said chamber in the loaded position is greater than 50% of the volume of said chamber.

Advantageously, the radial diameter of said reservoir is greater than its axial height.

Advantageously, said receiving sleeve comprises on its outer surface a visual mark, for example a coloured line that can be seen by the user when said reservoir is in the non-loaded position, and that cannot be seen when said reservoir is in the loaded position.

These features and advantages and others of the present invention will appear more clearly during the following detailed description, made in reference to the accompanying drawings, given as non-limiting examples, and in which

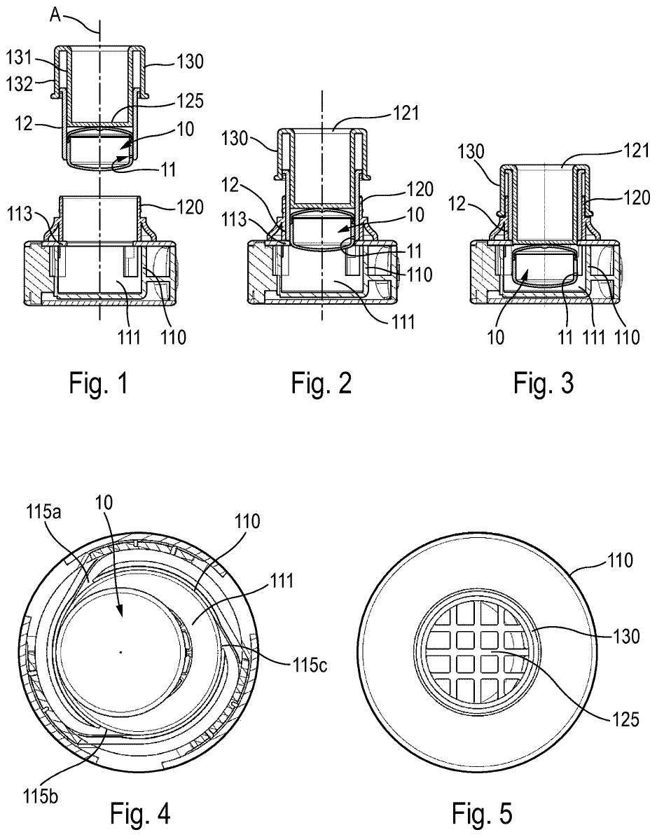

FIG. 1 is a diagrammatic section view of an inhaler in an advantageous embodiment, before inserting the reservoir in the inhaler;

FIG. 2 is a view similar to the view in FIG. 1, after assembling the reservoir, but in the non-loaded position.

FIG. 3 is a view similar to that of FIG. 2, in the loaded position,

FIG. 4 is a diagrammatic view from below of the reservoir in the chamber, in the loaded position, and

FIG. 5 is a diagrammatic view from above of the mouthpiece, in the loaded position.

In the description below, the terms “upper”, “lower” and “lateral” are with respect to the upright position of the device shown in FIGS. 1 to 3. The terms “axial” and “radial” refer to the longitudinal central axis A of the device, represented in FIG. 1.

In the embodiment shown in the drawings, the inhaler comprises a body 110 containing a chamber 111 and a receiving sleeve 120 adapted to receive a removable reservoir unit. Preferably, the chamber 111 is disposed between an inhalation air inlet and said receiving mouthpiece 120, in a manner such that when the user inhales, the air flow will pass through said chamber 111.

The reservoir unit comprises a reservoir 10, a closure ring 12 and an actuator member 130, forming a mouthpiece and defining the dispensing orifice 121.

The reservoir 10 contains a dose of dry powder to be inhaled and comprises a lateral outlet opening 11. The reservoir 10 is adapted to be disposed in an axially displaceable manner in the inhaler between an non-loaded position, shown in FIG. 2, and a loaded position, shown in FIG. 3. In the non-loaded position, the reservoir 10 is at least partially disposed outside said chamber 111, being positioned in the receiving sleeve 120. In the loaded position, it is entirely disposed in said chamber 111.

The reservoir 10 is inserted into the closure ring 12 adapted to close said lateral outlet opening 11. This insertion is preferably carried out by force-fitting, such that the reservoir 10 is retained by friction in said closure ring 12. This closure ring 12 thus protects the powder contained in the reservoir 10 until it is dispensed by actuating the inhaler. The closure ring 12 is displaceable and/or deformable with respect to the reservoir 10 between closed and open positions of the lateral outlet orifice 11, said closure ring 12 being in the closed position when said reservoir 10 is in the non-loaded position, and being in the open position when said reservoir 10 is in the loaded position.

The closing ring 12 is fixed in an axially displaceable manner in the actuator member 130, which serves to move said reservoir 10 from its non-loaded position towards its loaded position. This actuator member 130 is axially displaceable with respect to the body 110 in order to push the reservoir 10 from its non-loaded position towards its loaded position. Advantageously, the actuator member 130 comprises two coaxial walls 131, 132 between which said closure ring 12 can slide between its closed and open positions. Advantageously, as can be seen in FIG. 5, the actuator member 130 comprises a grid 125 upstream from the dispensing orifice 121, in contact with the reservoir 10.

Thus, before inserting the reservoir unit into the inhaler, the reservoir 10 is disposed in the closure ring 12 which is in the closed position.

When the reservoir unit is inserted into the inhaler, the lower edge of the closure ring 12 abuts against a radial projection 113 of the body 110. The reservoir 10 is then in the non-loaded position, with the lateral outlet opening 11 still closed by the closure ring 12.

When the user wishes to use the inhaler, the user moves the actuator member 130 axially downwards relative to the receiving sleeve 112 to push the reservoir 10 from its non-loaded position towards its loaded position. Since the closure ring 12 is axially blocked by the radial projection 113, the reservoir 10 is extracted from said closure ring 12 to come into the chamber 111, in the loaded position, with the lateral outlet opening 11 open.

Advantageously, the receiving sleeve 120 may comprise on its outer surface a visual mark, for example a coloured line that can be seen by the user when the reservoir 10 is in the non-loaded position, and that cannot be seen when said reservoir is in the loaded position. This makes it possible to inform the user that the actuator member 130 has been moved correctly. Thus, as long as the visual mark remains visible, the user knows that the reservoir 10 is not yet in the loaded position, and that he must not inhale, and it is only when the visual mark is no longer visible, being masked by the actuator member 130, that the user will inhale.

The diameter of the reservoir 10 is less than the diameter of said chamber 111, in a manner such that in the loaded position, the reservoir 10 can be displaced in the chamber 111. Advantageously, tangential channels 115a, 115b, 115c bring said flow of inhalation air into the chamber 111 in a tangential manner, thereby bringing about an orbital displacement of the reservoir 10 in the chamber 111, the reservoir 10 rotating on itself while rotating about the periphery of the chamber 111 along the lateral wall thereof. This movement will allow the powder contained in the reservoir 10 to be expelled, which is entrained towards the mouthpiece and the dispensing orifice by the flow of inhalation air.

Advantageously, the reservoir 10 may contain a dose of powder greater than 50 mg, in particular greater than 100 mg. Optionally, very large doses may be envisaged, in particular greater than 500 mg.

Advantageously, the volume occupied by the reservoir 10 in the chamber 111 in the loaded position is greater than 50% of the volume of the chamber 111.

Advantageously, the radial diameter of the reservoir 10 is greater than its axial height.

Advantageously, the reservoir 10 is formed by an upper reservoir portion and a lower reservoir portion, which are assembled after filling with the dose of powder. Advantageously, it is the lower reservoir portion which comprises the lateral outlet opening 11.

An advantage of the reservoir unit is that it is not symmetrical, which forms a foolproof key for the insertion of the reservoir 10 into the body 110 of the inhaler, this insertion only being possible in a single orientation of the reservoir 10. This is particularly advantageous when the external shape of the reservoir 10 is substantially symmetrical, but the lateral outlet orifice 11 is not disposed at the axial centre of the reservoir 10. In fact, in this case, it is desirable to correctly orientate the reservoir 10 in the inhaler for optimal operation, and the asymmetrical shape of the reservoir unit makes it possible to fulfil this function.

The device of the invention is simple and effective. It is made up of a small number of parts and is thus inexpensive to manufacture and to assemble, and is reliable in use. It allows optimal dispensing of the powder due to the orbital movement of the reservoir during actuation, while guaranteeing the integrity of the powder until it is used.

It should be noted that the inhaler can be refilled very easily, by replacing the reservoir unit with an empty reservoir with a reservoir unit with a full reservoir. Thus, the grid 125 and the actuator member 130 forming the mouthpiece are replaced after each use of the inhaler, together with the reservoir, such that there is no risk of pollution or contamination of the inhaler following the previous use.

Various modifications can also be envisaged by a person skilled in the art, without going beyond the ambit of the present invention, as defined by the accompanying claims.

1. A dry-powder inhaler, comprising:
a body containing a chamber and a receiving sleeve,
a reservoir unit intended to be removably inserted into said receiving sleeve, said reservoir unit comprising:
(i) a reservoir containing a dose of dry powder to be inhaled and comprising a lateral outlet opening, said reservoir being movable in said inhaler between a non-loaded position in which it is disposed in said receiving sleeve and a loaded position in which it is disposed entirely in said chamber,
(ii) a closure ring movable relative to said reservoir between a closed position of said lateral outlet opening and an open position of said lateral outlet opening, said closure ring being in a closed position when said reservoir is in the non-loaded position, and being in an open position when said reservoir is in the loaded position,
(iii) an actuator member forming a mouthpiece, for moving said reservoir from its non-loaded position to its loaded position, said actuator member defining a dispensing orifice of said inhaler.

2. The inhaler according to claim 1, wherein said actuator member comprises a grid upstream from said dispensing orifice.

3. The inhaler according to claim wherein said chamber is disposed between an inhalation air inlet and said receiving sleeve.

4. The inhaler as claimed in claim 1, wherein the diameter of said reservoir is less than the diameter of said chamber.

5. The inhaler as claimed in claim 1, wherein tangential channels bring a flow of inhalation air into said chamber in a tangential manner, which generates an orbital displacement of said reservoir in said chamber.

6. The inhaler as claimed in claim 1, wherein said reservoir contains a dose of powder greater than 50 mg, in particular greater than 100 mg.

7. The inhaler as claimed in claim 1, in which the volume occupied by said reservoir in said chamber in the loaded position is greater than 50% of the volume of said chamber.

8. The inhaler as claimed in claim 1, in which the radial diameter of said reservoir is greater than its axial height.

9. The inhaler as claimed in claim 1, wherein said receiving sleeve comprises on its outer surface a visual mark, for example a coloured line that can be seen by the user when said reservoir is in the non-loaded position, and that cannot be seen when said reservoir is in the loaded position.
